(19) United States
(12) Patent Application Publication
(10) Pub. No.: US 2020/0368382 A1
(43) Pub. Date: Nov. 26, 2020

(54) FRAGRANCE RELEASE COMPOSITION

(71) Applicant: SYMRISE AG, Holzminden (DE)

(72) Inventors: Jörg DRÖGE, Bodenwerder (DE); Ranjita SHEGOKAR, Goch (DE)

(21) Appl. No.: 16/638,511

(22) PCT Filed: Aug. 18, 2017
(86) PCT No.: PCT/EP2017/070951

§ 371 Date: Feb. 12, 2020

(51) Int. Cl. A61L 9/01; C11D 3/50

(52) U.S. Cl. CPC A61L 9/01; C11D 3/50

(57) ABSTRACT

The present invention relates to a fragrance release composition comprising at least two fillers, one or more consistency builder(s), one or more surfactant(s) and a fragrance. In particular, the present invention relates to a fragrance release composition that can be prepared and shaped, preferably extruded, in a variety of shapes at various temperatures including room temperature but also when heated up. Furthermore, the present invention relates to a product comprising a fragrance release composition according to the invention and a method for producing such a fragrance release composition or product as well as its use to impart fragrance to air or to fabric and combination products.

The present invention relates to a fragrance release composition comprising at least two fillers, one or more consistency builder(s), one or more surfactant(s) and a fragrance. In particular, the present invention relates to a fragrance release composition that can be prepared and shaped, preferably extruded, in a variety of shapes at various temperatures including room temperature but also when heated up. Furthermore, the present invention relates to a product comprising a fragrance release composition according to the invention and a method for producing such a fragrance release composition or product as well as its use to impart fragrance to air or to fabric and combination products.

Scent boosters are products, which are used to give off a scent for example to fabric such as clothes during laundry or in order to freshen the air in a room or other enclosed spaces such as the inside of a car, a closet or a dishwasher or to impart a fragrance to an object. Conventional scent boosters are prepared on the basis of melted polyethylene glycol (PEG), which is formed into specific shapes, for example provided in particulate form to be used as a washing additive. The particles, usually beads or pastilles of a few millimeters in diameter, dissolve quickly during the laundry cycle and impart as scent to the laundry, which ideally remains after washing and drying for up to several days. On the other hand, such compositions may also give off their scent over an extended period of time without being dissolved in water but merely left in contact with air and thus be used as air fresheners.

The production of conventional scent boosters, is expensive, time consuming and limited with respect to ingredient choice and the requirement of special equipment. In order to provide the product in the desired shape, the base composition has to be heated up above the melting temperature to be mixed homogeneously, extruded or otherwise pressed into a form and then cooled before it can be packaged and used. A suitable base composition usually comprises expensive ingredients and the procedure including heating and cooling takes time and requires special equipment, which is costly to purchase and maintain. Furthermore, many fragrances tend to evaporate quickly at higher temperatures and therefore the fragrance content of the composition decreases rapidly during production.

It would therefore be desirable, to have a composition, which is composed of inexpensive materials and can be shaped at various temperatures including room temperature, while simultaneously being solid enough so as to not lose its shape over time. In addition, such a composition should have suitable properties with respect to dissolving time in water and fragrance release over time according to the desired application.

It was therefore an objective of the present invention to provide a composition capable of releasing a fragrance and imparting a scent to the air or to an object, in particular to a fabric, which can be produced and formed into a desired shape at room temperature.

Furthermore, it was an object of the present convention that the composition is composed of inexpensive materials and has favorable properties with respect to its dissolution and fragrance release behavior.

The objectives of the present invention are met by a fragrance release composition comprising or consisting of:
i) at least two fillers,
ii) one or more consistency builder(s),
iii) one or more surfactant(s), and
iv) a fragrance or mixture of two or more fragrances.

It has been found out in the context of the present invention, that it is possible to provide a fragrance release composition that consists of inexpensive materials and can be produced and formed into a desired shape at room temperature. The components of the composition will be explained in detail below.

A fragrance release composition in the context of the present invention is a composition comprising a base material, which can be formed into a desired shape, and a fragrance or fragrance mixture dispersed in the base material which is released quickly either when the base material comes into contact with water and is fully or partially dissolved or over an extended period of time when the composition is left in contact with air.

The composition according to the present invention comprises two fillers. Fillers are inexpensive materials that serve to add bulk to a material while at the same time not changing the overall desired consistency, structure or other properties of the material. It has been found out in the trials leading up to the present invention, that all the above identified requirements can best be fulfilled simultaneously when a combination two different filler materials is used together. Examples of suitable fillers and combinations of fillers are given below.

Furthermore, the composition according to the present invention comprises one or more consistency builder(s). A consistency builder is the context of the present invention is a material, which, depending on the overall composition, acts as a binder or a plasticizer but can also improve the dissolution behavior of the composition. Suitable materials to be used as consistency builders are given below.

The one or more surfactant(s) in the composition according to the present invention allow a homogenous mixture of the components and may also add cleaning properties to the composition, which, however, is not the main function. The surfactant(s) should also be inexpensive and provide optimal emulsifying properties, e.g. when a perfume oil is used as fragrance in the otherwise water-soluble composition, but should at the same time not influence the overall consistency and other desired properties of the composition. Conventional surfactants can be used, but for an optimized performance, two different surfactants provide even better results as detailed below.

According to a preferred embodiment, the composition therefore comprises two surfactants. Examples of surfactants and combinations are given below.

The fragrance or fragrance mixture to be released is not particularly limited and can also comprise raw fragrance materials or malodor actives. Particularly preferred are perfume oils. Further examples are given below.

According to a preferred embodiment, the fragrance release composition comprises or consists of
i) 20 to 70 wt.-%, preferably 30 to 60 wt. % of at least two fillers,
ii) 10 to 60 wt.-%, preferably 20 to 50 wt.-% of one or more consistency builder(s),
iii) 2 to 30 wt.-%, preferably 5 to 20 wt.-% of one or more surfactant(s), and
iv) 1 to 30 wt.-%, preferably 2 to 20 wt.-% of a fragrance or mixture of two or more fragrances.

In the composition according to the present invention, the consistency builder(s) represent at most 60 wt. % and can largely be replaced by inexpensive fillers making the composition less costly compared to conventional scent booster compositions. Moreover, the proportion of components leads to a consistency, which allows production at room temperature without time and energy consuming heating and cooling steps or expensive equipment. Therefore, the composition according to the present invention can be produced at much lower expense in total.

In a preferred embodiment, the at least two fillers are selected from the group consisting of carbohydrates, in particular sugar, inverted sugar, mono-, di- and oligosaccharides, cellulose, native starch, gelatinized starch, pre-gelatinized starch, mannitol and lactose, and salts, in particular sodium sulfate, sodium chloride, calcium magnesium carbonate, silicates and dicalcium phosphate, preferably the at least two fillers comprise a carbohydrate and a salt, in particular a disaccharide and a salt.

In principle, any conventional fillers may be used. It has been found out, however, that a combination of a carbohydrate, in particular a disaccharide and a salt provides the best performance with respect to adding bulk by using inexpensive materials and at the same time not influencing the properties in such a way that the desirable features of the composition, i.e. workability at room temperature and dissolution as well as fragrance release behavior, are lost.

“Sugar” as used herein refers to sucrose. Specifically refined industry grade sugar can be used. Instead of sugar, also honey can be used as replacement.

According to a further preferred embodiment, the consistency builder(s) is/are selected from the group consisting of polyethylene glycols, low melting point lipids and polyvinyl pyrrolidone, preferably polyethylene glycols.

The consistency builder(s) in the composition according to the present invention serve as binder(s) and as plasticizer(s) to adjust the composition to the optimal consistency, which allows processing at room temperature. If the composition is too hard or too brittle it can not be formed into the desired shape at room temperature. If it is too soft, on the other hand, it will not keep it's shape around ambient temperatures, in which it may be used.

Suitable consistency builders are low melting point lipids and, in particular, polyethylene glycols, which can be used at different molecular weights to provide the desired properties. Preferred are polyethylene glycols with a molecular weight in a range from 1 000 to 20 000 g/mol, particularly preferred 4 000 to 10 000 g/mol. Polyethylene glycols have the added advantage that they promote quick disintegration upon contact with water, i.e. improve the dissolving behavior of the composition as they are water soluble.

Preferably, the surfactant(s) is/are selected from the group consisting of ceteareth 20, sodium alkylbenzene sulphonate, sodium lauryl sulfate, sodium lauryl ether sulfate, secondary alkane sulfonate, sodium cholate, cetyltrimethyl ammonium bromide, lauryl dimethyl amine oxide, lecithin, polyoxyethylene fatty acid amides, glycol esters of fatty acids, preferably ceteareth 20 and sodium alkylbenzene sulfonate.

The surfactant(s) in the composition of the present invention allow formulation of a homogenous composition, in which the fragrance is evenly dispersed, without negatively affecting the consistency. In addition, they may provide cleaning properties as a side effect. Any conventional surfactants can be used alone or in combination. It has been found out in the experimental trials leading up to the present invention, that a combination of ceteareth 20 and sodium alkylbenzene sulfonate, preferably in equal amounts, provides the best performance according to the above criteria including price.

Ceteareth-20 (CAS #68439-49-6) is the polyethylene glycol ether of cetearyl alcohol wherein the number 20 indicates the average number of ethylene oxide residues in the polyoxyethylene chain.

Particularly preferred is a fragrance composition as described above, in which the fillers comprise or consist of a salt, preferably sodium sulfate, and a disaccharide, preferably sugar and/or wherein the surfactants comprise or consist of ceteareth 20 and sodium alkylbenzene sulfonate.

Especially preferred is an embodiment, in which the fillers comprise or consist of a salt, preferably sodium sulfate, and a disaccharide, preferably sugar and/or wherein the surfactants comprise or consist of ceteareth 20 and sodium alkylbenzene sulfonate and/or wherein the consistency builder(s) comprise or consist of polyethylene glycol.

According to a preferred embodiment, the fragrance composition comprises or consist of:
ia) 5 to 50 wt.-%, preferably 10 to 30 wt.-%, particularly preferably 15 to 25 wt.-% of a salt, preferably sodium sulfate,
ib) 10 to 60 wt. %, preferably 15 to 50, particularly preferably 20 to 40 wt.-% of a disaccharide, preferably sugar,
ii) 10 to 60 wt.-%, preferably 20 to 50, particularly preferably 30 to 40 wt.-% of polyethylene glycol,
iiia) 1 to 15 wt-%, preferably 2 to 10 wt.-%, particularly preferably 3 to 7 wt.-% ceteareth 20,
iiib) 1 to 15 wt-%, preferably 2 to 10 wt.-%, particularly preferably 3 to 7 wt.-% sodium alkylbenzene sulphonate, and
iv) 1 to 30 wt.-%, preferably 2 to 15 wt.-%, particularly preferably 2 to 7 wt.-% of a fragrance or mixture of two or more fragrances.

The composition comprising or consisting of the above defined components in the recited amounts has been found to have the optimal consistency to be processed at room temperature, comprise largely inexpensive materials, be stable enough to maintain its shape and give off the fragrance over an extended period of time into the air and dissolve quickly upon contact with water.

The fragrances that can be used in the composition of the present invention are not particularly limited. Suitable fragrances may for example be selected from anethole, benzaldehyde, benzyl acetate, benzyl alcohol, benzyl formate, iso-bornyl acetate, camphene, ciscitral (neral), citronellal, citronellol, citronellyl acetate, p-cymene, decanal, dihydrolinalool, dihydromyrcenol, dimethyl phenyl carbinol, eucaliptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, mycrene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, and vertenex (p-tertiary-butyl cyclohexyl acetate), amyl cinnamic aldehyde, isoamyl salicylate, beta-caryophyllene, cedrene, cinnamic alcohol, coumarin, dimethyl benzyl carbinyl acetate, ethyl vanillin, eugenol, isoeugenol, flor acetate, heliotropine, 3-cis-hexenyl salicylate, hexyl salicylate, filial (p-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, and veratraldehyde. Cedarwood terpenes are composed mainly of alpha-cedrene, beta-cedrene, and other C15H24 sesquiterpenes, benzophenone, benzyl salicylate, ethylene brassylate, galaxolide (1,3,4,6,7,8-hexahydro-4,6,6,7,8,8-hexamethyl-cyclopenta-gama-2-benzopyran), hexyl cinnamic aldehyde, lyral (4-(4-hydroxy-4-methyl pentyl)-3-cyclohexene-10-carboxaldehyde), methyl cedrylone, methyl dihydro jasmonate, methyl-beta-naphthyl ketone, musk indanone, musk ketone, musk tibetene, and phenylethyl phenyl acetate, angelica root oil, anise oil, arnica flower oil, basil oil, bay oil, champaca flower oil, silver fir oil, silver fir cone oil, elemi oil, eucalyptus oil, fennel oil, spruce needle oil, galbanum oil, geranium oil, ginger grass oil, guaiacwood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lemongrass oil, musk seed oil, myrrh oil, clove oil, neroli oil, niaouli oil, olibanum oil, oregano oil, palmarosa oil, patchouli oil, Peru balsam oil, petitgrain oil, pepper oil, peppermint oil, pimento oil, pine oil, rosemary oil, sandalwood oil, celery oil, star anise oil, thuja oil, thyme oil, verbena oil, vetiver oil, juniper berry oil, wormwood oil, wintergreen oil, ylang-ylang oil, hyssop oil, cinnamon oil, cinnamon leaf oil and cypress oil, ambrettolide, α-amyl cinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amyl ketone, methylanthranilic acid methyl ester, p-methyl acetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenylethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratrum aldehyde, cinnamaldehyde, cinnamyl alcohol, cinnamic acid, cinnamic acid ethyl ester and cinnamic acid benzyl ester, alkyl isothiocyanates (alkyl mustard oils), buta-nedione, limonene, linalool, linalyl acetate, linalyl propionate, menthol, menthone, methyl-n-heptenone, phellandrene, phenylacetaldehyde, terpinyl acetate, citral and citronellal.

The above recited fragrances are only given by way of examples and any other fragrance, fragrance raw material or combination of two or more fragrances can be used. The fragrance or one of the fragrances can also be provided in the form of (micro)capsules, e.g. as encapsulated fragrance oil in a polymer or lipid shell or a combination thereof.

The fragrance release composition described above can be shaped, in particular extruded, at room temperature in various shapes and preferably provided in particulate form. It may comprise or consist of particles in one or more shape(s) selected from spheres, beads, half beads, pastilles, pellets, discs or petals and the particles may have an average diameter of 1 to 20 mm, preferably 3 to 15 mm, particularly preferably 5 to 10 mm.

Room temperature in the context of the present invention refers to 25° C. However, the composition according to the invention maintains its consistency and workability also at temperatures around room temperatures, i.e. at least within a range from 20° C. to 30° C.

Advantageously, the composition according to the present invention in particulate form as described above dissolves in water in less than 30 minutes, preferably less than 20 minutes, particularly preferably in less than 18 minutes.

A further preferred embodiment of the present invention is a product comprising or consisting of a fragrance release composition as described above, in particular wherein the product is a washing additive or an air freshener.

A product according to the present invention may consist of the composition as described above in particulate or compact form or it may comprise the composition for example as an additive or it may be provided with a means to place it at the location where it is used and provide contact with air or water as desired. For example it can be provided in a casing having holes, which may be attached to an object, such as a toilet or a dishwasher at a desired location.

A washing additive is preferably used in combination with laundry or dish soap during washing cycles of a washing machine or a dishwasher or when washing by hand. Preferably it imparts a scent to the washing goods, i.e. the laundry. Air fresheners give off a scent over an extended amount of time, preferably several days, when they are left in contact with air and/or they release the fragrance more quickly when they come into contact with water as the base material starts to dissolve, e.g. in a toilet rim block. Air freshening may be desired in a room or an enclosed space such as the inside of a closet or a car or a dishwasher. In case the composition is used in a dishwasher it may advantageously be provided in food grade as only food grade components can be employed.

The present invention also relates to a method for producing a fragrance release composition or a product as described above comprising the steps:
a) providing the components i) to iv) as defined above,
b) combining the components provided in step a),
c) mixing the combined components to obtain a homogenous mixture,
d) shaping, in particular extruding, the homogenous mixture obtained in step c) at a temperature from 10 to 80° C., preferably from 15 to 50° C., particularly preferably from 20 to 30° C., and
e) optionally processing and/or packaging the shaped mixture.

Advantageously, the composition described above can be produced at room temperature and it is neither necessary to heat it up above a melting temperature to homogenously mix it nor to shape it into the desired form and cool it down again. Therefore, the time and energy consumption of the production process is very low. However, if for some reason desirable, the composition may also be processed at much higher temperatures without losing its desirable properties.

The shaping of the mixture in step d) can be done by passing the mass through an opening of desired shape and size or pressing it into a form. Furthermore, shaping can be achieved by wet or dry granulation optionally followed by tabletting. Preferably, the mixture is extruded.

The processing in step e) may include cutting the extruded or pressed form in a desired size, dedusting it e.g. with an aerosol, covering with an agent to prevent sticking together of single particles (e.g. Aerosil 200) or any other lubricating agent and/or packaging in a desired form, e.g. in an air tight container for storage or shipping.

The present invention also relates to a fragrance release composition or product obtained or obtainable by a method as described above. Such a composition or product has the properties and advantages as described above.

Finally, the present invention also relates to the use of a fragrance release composition or product as described above to impart a fragrance to air or to fabric.

The composition according to the present invention can be used to give off a scent in the air, in particular an enclosed space such as the inside of a car, a closet or a dishwasher or it can be used to impart a fragrance to an object, in particular to fabric such as clothes during laundry when used as a washing additive. The scent advantageously remains on the fabric for up to several days. A washing additive is preferably used in combination with laundry or dish soap during washing cycles of a washing machine or a dish washer. When used as an air freshener, the composition according to the present invention gives off a scent over an extended amount of time, preferably several days, when it is left in contact with air and/or it releases the fragrance more quickly when it comes into contact with water as the base material starts to dissolve, e.g. in a toilet rim block. In case the composition is used in a dishwasher it may advantageously be provided in food grade as only food grade components can be employed.

### EXAMPLE 1: PREPARATION OF A FRAGRANCE RELEASE COMPOSITION IN DISC FORM

The following ingredients are combined:

| | |
|---|---|
| Sodium sulfate | 20.00 |
| Hoesch TG 20 | 5.00 |
| Lipoxol 6000 | 35.00 |
| Marlon ARL | 5.00 |
| Laundry perfume | 5.00 |
| Sugar | 30.00 |
| | 100.00 |

The ingredients are mixed at 25° C. to provide a homogenous mixture. The mixture is fed into an extruder and extruded at 25° C. to give a rod having a diameter of 5 mm. The rod is cut into discs of 3 mm width.

### EXAMPLE 2: DISSOLVING TIME IN WATER

The discs of example 1 and discs of the same size but having a conventional polyethylene glycol base and a fragrance are dissolved in water. Both discs dissolve completely within 16 mins.

1. A fragrance release composition comprising:
i) at least two fillers,
ii) one or more consistency builder(s),
iii) one or more surfactant(s), and
iv) a fragrance or mixture of two or more fragrances.

2. The fragrance release composition according to claim 1, comprising:
i) 20 to 70 wt.-% of at least two fillers,
ii) 10 to 60 wt.-% of one or more consistency builder(s),
iii) 2 to 30 wt.-% of one or more surfactant(s), and
iv) 1 to 30 wt.-% of a fragrance or mixture of two or more fragrances.

3. The fragrance release composition according to claim 1, wherein the at least two fillers are selected from carbohydrates and salts.

4. The fragrance release composition according to claim 1, wherein the consistency builder(s) is/are selected from the group consisting of polyethylene glycols, low melting point lipids, and polyvinyl pyrrolidone.

5. The fragrance release composition according claim 1, wherein the surfactant(s) is/are selected from the group consisting of ceteareth 20, sodium alkylbenzene sulphonate, sodium lauryl sulfate, sodium lauryl ether sulfate, secondary alkane sulfonate, sodium cholate, cetyltrimethyl ammonium bromide, lauryl dimethyl amine oxide, lecithin, polyoxyethylene fatty acid amides, glycol esters of fatty acids, preferably ceteareth 20, and sodium alkylbenzene sulfonate.

6. The fragrance release composition according to claim 3, wherein the fillers comprise a salt and a disaccharide.

7. The fragrance release composition according to claim 1, wherein the surfactants comprise ceteareth 20 and sodium alkylbenzene sulphonate.

8. The fragrance release composition according claim 2 comprising:
ia) 5 to 50 wt.-% of a salt,
ib) 10 to 60 wt. % of a disaccharide,
ii) 10 to 60 wt.-% of polyethylene glycol,
iiia) 1 to 15 wt.-% of ceteareth 20,
iiib) 1 to 15 wt.-% of sodium alkylbenzene sulphonate, and
iv) 1 to 30 wt.-% of a fragrance or mixture of two or more fragrances.

9. The fragrance release composition according to claim 1, wherein the composition is formulated to be extruded.

10. The fragrance release composition according to claim 1, wherein the composition is in the form of particles, the particles having a shape selected from spheres, beads, half beads, pastilles, pellets, discs, petals, and combinations thereof.

11. The fragrance release composition according to claim 10, wherein the particles have an average diameter of 1 to 20 mm.

12. A product comprising:
a fragrance release composition according to claim 1, wherein the product is a washing additive or an air freshener.

13. A method for producing the fragrance release composition according to claim 1 comprising:
a) providing the components i) to iv),
b) combining the components provided in step a),
c) mixing the combined components to obtain a homogenous mixture,
d) shaping the homogenous mixture obtained in step c) at a temperature from 10 to 80° C., and
e) optionally, processing and/or packaging the shaped mixture.

14. (canceled)

15. (canceled)

16. The fragrance release composition according to claim 3 comprising one or more carbohydrates selected from sugar, inverted sugar, mono-, di- and oligosaccharides, cellulose, native starch, gelatinized starch, pre-gelatinized starch, mannitol and lactose, and wherein, if present, the salt is selected from sodium sulfate, sodium chloride, calcium magnesium carbonate, silicates, dicalcium phosphate, and a combination thereof.

17. The fragrance release composition according to claim 6, wherein the at least two fillers comprise sodium sulfate and sugar.

18. The fragrance release composition according to claim 8 comprising:
ia) 15 to 25 wt. % of a salt,
ib) 20 to 40 wt.-% of a disaccharide,
ii) 30 to 40 wt. % of polyethylene glycol,
iiia) 3 to 7 wt.-% of ceteareth 20,
iiib) 3 to 7 wt.-% of sodium alkylbenzene sulphonate, and
iv) 2 to 7 wt.-% of a fragrance or mixture of two or more fragrances.

19. The fragrance release composition according to claim 9, wherein the composition is formulated to be extruded into a particulate form.

20. A method for imparting a fragrance to air or to fabric comprising:
(a) mixing the following components:
i) at least two fillers,
ii) one or more consistency builder(s),
iii) one or more surfactant(s), and
iv) a fragrance or mixture of two or more fragrances.

21. The method of claim 20 further comprising:
(b) applying the mixture of (a) to a fabric.

22. The method of claim 20, wherein the at least two fillers are selected from carbohydrates and salts.
